(19) United States
(12) Patent Application Publication
(10) Pub. No.: US 2003/0115686 A1
(43) Pub. Date: Jun. 26, 2003

(54) Hair relaxer or straightener without lye or drying effect

(76) Inventor: Gary Grey, New York, NY (US)

Correspondence Address: OSTROLENK FABER GERB & SOFFEN, 1180 AVENUE OF THE AMERICAS, NEW YORK, NY, 100368403

(21) Appl. No.: 10/329,975

(22) Filed: Dec. 23, 2002

Related U.S. Application Data
(60) Provisional application No. 60/342,334, filed on Dec. 22, 2001.

(51) Int. Cl. A61K 7/13

(52) U.S. Cl. 8/405; 8/406

(57) ABSTRACT

A hair relaxer comprised of an activator that includes guanidine hydrochloride in place of guanidine carbonate as the activator mixed in a calcium hydroxide based relaxer.

### CROSS-REFERENCE TO RELATED APPLICATION

This application is based on and claims priority to provisional application No. 60/342,334, filed Dec. 21, 2001, herein incorporated by reference.

### BACKGROUND OF THE INVENTION

The present invention relates to a hair relaxer or straightener.

Hair relaxers or straighteners tend to reduce the curl or waviness in the hair. There are many hair relaxers on the market.

Most hair relaxers (straighteners) on the market use sodium hydroxide or calcium hydroxide, in a reaction with guanidine carbonate as their active ingredients. Sodium relaxers are very harsh and may be damaging to the hair and scalp. Calcium relaxers have other problems. While they are more gentle than sodium relaxers, the reaction between calcium hydroxide and guanidine carbonate produces calcium carbonate which deposits on the hair and which also has a severe drying effect on the hair over time. A relaxer including lye would have a severe drying effect.

The are other types of relaxers on the market. One type includes the use of lithium hydroxide which has a poor shelf life. Another type includes potassium hydroxide which functions almost exactly like sodium hydroxide. But it is more expensive, so it is rarely used.

U.S. Pat. No. 4,314,572 discloses the use of guanidine hydrochloride along with a hydroxide form to form guanidine hydrochloride which is then purified and used for hair treatment. Guanidine hydrochloride is applied to the hair.

U.S. Pat. Nos. 4,416,296 and 6,007,585 disclose a composition for hair treatment comprising guanidine salts with inorganic hydroxides such as calcium hydroxide. However, these references do not disclose guanidine hydrochloride as the salt to be used.

### SUMMARY OF THE INVENTION

This invention proposes to use guanidine hydrochloride instead of conventionally used guanidine carbonate as the “activator” in a calcium hydroxide based relaxer. When guanidine hydrochloride reacts with calcium hydroxide, the resulting product is calcium chloride, which is a salt, and is water soluble. Hence, there is no drying effect on the hair.

Guanidine hydrochloride is a well known substance listed in the Merck Index. It is not known for use as a hair relaxer.

An example of a final product embodying the invention may consist of a cream base and an activator. The cream base and the activator are kept separated prior to use and a user would mix them together shortly before use t activate the relaxer to relax the hair.

### DESCRIPTION OF A PREFERRED EMBODIMENT

An example of the activator formulation according to the invention, as known to the inventor hereof now, consists essentially of:

Water (Q.S.) (Solvent.)

Guanidine Hydrochloride (15-30%) (Active ingredient, reacts with calcium hydroxide in cream base)

Xantham Gum (0.1-0.5%) (Thickener.)

An example of the cream base formulation to be mixed together with the activator formulation according to the invention, as known to the inventor now, consists essentially of:

Emulsifying Wax (7-12%) (Thickener, emulsifier.)

Cetyl Alcohol (1-3%) (Viscosity modifier, co-emulsifier)

Mineral Oil (8-15%) (Emulsion formation.)

Petrolatum (5-13%) (Emulsion formation.)

Propylene Glycol (3-5%) (Humectant.)

Calcium Hydroxide (5-9%) (Active ingredient, reacts with guanidine hydrochloride.)

Water (Q.S.) (Solvent, and emulsion formation.)

Although the present invention has been described in relation to particular embodiments thereof, many other variations and modifications and other uses will become apparent to those skilled in the art. Therefore, the present invention is not limited by the specific disclosure herein.

1. A hair relaxer or straightener comprising a calcium hydroxide based relaxer combined with an activator salt of guanidine hydrochloride.

2. The relaxer of claim 1, wherein the calcium hydroxide is included in a cream base formulation and the guanidine hydrochloride is in an activator that is mixed into the cream based formulation to produce the relaxer.

3. The relaxer of claim 2, wherein the activator is in a solvent.

4. The relaxer of claim 3, wherein the solvent is water.

5. The relaxer of claim 3, wherein the guanidine hydrochloride is in an amount of 15-30% of the activator formulation and the calcium hydroxide is between 5-9% of the cream base formulation.

6. The hair relaxer of claim 1, wherein the activator consists essentially of:
Water (Q.S.) (Solvent.)
Guanidine Hydrochloride (15-30%) (Active ingredient, reacts with calcium hydroxide in cream base)
Xantham Gum (0.1-0.5%) (Thickener.)

7. The hair relaxer of claim 6, wherein the cream base formulation consists essentially of:
Emulsifying Wax (7-12%) (Thickener, emulsifier.)
Cetyl Alcohol (1-3%) (Viscosity modifier, co-emulsifier)
Mineral Oil (8-15%) (Emulsion formation.)
Petrolatum (5-13%) (Emulsion formation.)
Propylene Glycol (3-5%) (Humectant.)
Calcium Hydroxide (5-9%) (Active ingredient, reacts with guanidine
hydrochloride.)
Water (Q.S.) (Solvent, and emulsion formation.)

8. The hair relaxer of claim 1, wherein the cream base formula consists essentially of:
Emulsifying Wax (7-12%) (Thickener, emulsifier.)
Cetyl Alcohol (1-3%) (Viscosity modifier, co-emulsifier)
Mineral Oil (8-15%) (Emulsion formation.)
Petrolatum (5-13%) (Emulsion formation.)
Propylene Glycol (3-5%) (Humectant.)
Calcium Hydroxide (5-9%) (Active ingredient, reacts with guanidine
hydrochloride.)
Water (Q.S.) (Solvent, and emulsion formation.)
